(19) United States
(12) Patent Application Publication
(10) Pub. No.: US 2008/0300506 A1
(43) Pub. Date: Dec. 4, 2008

(54) BIOPSY DEVICE WITH MULTIPLE CUTTERS

(75) Inventor: Jon T. McIntyre, Newtonville, MA (US)

Correspondence Address: Vista IP Law Group LLP, 2040 MAIN STREET, 9TH FLOOR, IRVINE, CA, 92614, US

(73) Assignee: BOSTON SCIENTIFIC SCIMED, INC., Maple Grove, MN (US)

(21) Appl. No.: 12/126,822

(22) Filed: May 23, 2008

Related U.S. Application Data
(60) Provisional application No. 60/940,636, filed on May 29, 2007.

(51) Int. Cl. A61B 10/02

(52) U.S. Cl. 600/566; 600/567

(57) ABSTRACT

A tissue sampling device comprises an elongated tube having a distal end with at least two apertures. The device further comprises at least two cutters disposed within the elongated tube, wherein each cutter is configured to be independently displaced longitudinally within the elongated tube and across a respective one of the apertures to sever tissue prolapsed within the respective aperture. A method comprises inserting the device in tissue. The method further comprises receiving a first prolapsed portion of the tissue into the first aperture, longitudinally displacing the first cutter across the first aperture to sever the first prolapsed tissue portion within the first aperture, receiving a second prolapsed portion of the tissue into the second aperture, and longitudinally displacing the second cutter independently from the first cutter across the second aperture to sever the second prolapsed tissue portion within the second aperture.

### RELATED APPLICATION DATA

The present application claims the benefit under 35 U.S.C. § 119 to U.S. Provisional Patent Application Ser. No. 60/940,636, filed May 29, 2007. The foregoing application is incorporated by reference into the present application in its entirety for all purposes.

### FIELD OF THE INVENTION

The present invention is in the field of devices to obtain biopsy samples.

### BACKGROUND

The diagnosis and treatment of patients with cancerous tumors, premalignant conditions, and other disorders has long been an area of intense interest in the medical community. Non-invasive methods for examining tissue and, more particularly, breast tissue include palpation, X-ray imaging, MRI imaging, CT imaging, and ultrasound imaging. When a physician suspects that tissue may contain cancerous cells, a biopsy may be done using either an open procedure or in a percutaneous procedure. In an open procedure, a scalpel is used by the surgeon to create an incision to provide direct viewing and access to the tissue mass of interest. The biopsy may then be done by removal of the entire mass (excisional biopsy) or a part of the mass (incisional biopsy). In a percutaneous biopsy, a needle-like instrument is inserted through a very small incision to access the tissue mass of interest and to obtain a tissue sample for examination and analysis. The advantages of the percutaneous method as compared to the open method are significant: less recovery time for the patient, less pain, less surgical time, lower cost, less disruption of associated tissue and nerves and less disfigurement. Percutaneous methods are generally used in combination with imaging devices such as X-ray and ultrasound to allow the surgeon to locate the tissue mass and accurately position the biopsy instrument.

Generally there are two ways to percutaneously obtain a tissue sample from within the body, aspiration or core sampling. Aspiration of the tissue through a fine needle requires the tissue to be fragmented into small enough pieces to be withdrawn in a fluid medium. Application is less intrusive than other known sampling techniques, but one can only examine cells in the liquid (cytology) and not the cells and the structure (pathology). In core biopsy, a core or fragment of tissue is obtained for histologic examination which may be done via a frozen or paraffin section. The type of biopsy used depends mainly on various factors and no single procedure is ideal for all cases.

Typical biopsy needles are designed to obtain a single specimen per insertion. If more than one specimen is desired, the biopsy needle is removed and then reinserted. In addition to increased patient discomfort, the reinsertion of the biopsy needle adds time to the procedure and is inefficient.

### SUMMARY OF THE INVENTION

In accordance with a first aspect of the present inventions, a tissue sampling device comprises an elongated tube having a distal end with two apertures. The elongated tube may have a suitable size, such as in the range of 14 gauge to 7 gauge, and may take the form of a needle. In one embodiment, the apertures radially oppose each other, and each aperture circumferentially extends about the elongated tube greater than ninety degrees. The tissue sampling device further comprises two cutters disposed within the elongated tube, wherein each cutter is configured to be independently displaced longitudinally within the elongated tube and across a respective one of the apertures to sever tissue prolapsed within the respective aperture.

In one embodiment, each cutter has a semi-circular cross-section. In an optional embodiment, the tissue sampling device comprises a firing mechanism (e.g., a pneumatic or spring-loaded mechanism) configured to rapidly advance each cutter towards the distal end of the needle and across the respective aperture to sever the prolapsed tissue. In another optional embodiment, the tissue sampling device further comprises a cannula in which the elongated tube is coaxially housed. In still another optional embodiment, the elongated tube has one or more lumens in fluid communication with the apertures, in which case, the tissue sampling device may further comprise a vacuum port in fluid communication with the one or more lumens.

In accordance with a second aspect of the present inventions, a method of obtaining a tissue sample using a biopsy probe is provided. The biopsy probe comprising first and second apertures and first and second cutters. The method comprises inserting the biopsy probe in tissue (e.g., malignant tissue). In one method, the biopsy probe is inserted in the tissue while the first and second cutters respectively close the first and second apertures. The method further comprises receiving a first prolapsed portion of the tissue into the first aperture, longitudinally displacing the first cutter across the first aperture to sever the first prolapsed tissue portion within the first aperture, receiving a second prolapsed portion of the tissue into the second aperture, and longitudinally displacing the second cutter independently from the first cutter across the second aperture to sever the second prolapsed tissue portion within the second aperture.

In one method, the first and second cutters are independently longitudinally retracted to open the first and second apertures, thereby allowing the first and second prolapsed tissue portions to be received into the first and second apertures. In another method, the first and second prolapsed tissue portions are respectively vacuumed into the first and second apertures. In still another method, the first and second cutters are rapidly displaced across the first and second apertures to sever the respective first and second prolapsed tissue portion. The first and second severed tissue portions may be aspirated through the biopsy probe. An optional method comprises rotating the biopsy probe within the tissue (e.g., 90 degrees), receiving a third prolapsed portion of the tissue into the first aperture, longitudinally displacing the first cutter across the first aperture to sever the third prolapsed tissue portion within the first aperture, receiving a fourth prolapsed portion of the tissue into the second aperture, and longitudinally displacing the second cutter independently from the first cutter across the second aperture to sever the fourth prolapsed tissue portion within the second aperture.

Other and further aspects and features of the invention will be evident from reading the following detailed description of the preferred embodiments, which are intended to illustrate, not limit, the invention

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 shows an embodiment of a biopsy device;

FIG. 2 depicts the distal end of an embodiment of a needle of the biopsy device of FIG. 1;

FIG. 3 is an end cross-sectional view of the needle of FIG. 2, taken along line 3-3 as viewed in the direction of the arrows;

FIG. 4 depicts the needle of FIG. 2 in which one of the cutters is fully retracted.

FIG. 5 shows an embodiment of a spring-loaded firing mechanism;

FIG. 6 shows an embodiment of a pneumatic firing mechanism;

FIG. 7 shows another embodiment of a pneumatic firing mechanism;

FIG. 8A depicts the biopsy device of FIG. 1 in a ready to sample position;

FIG. 8B depicts the biopsy device of FIG. 1 where the needle is advanced into the tissue of interest;

FIG. 8C depicts the biopsy device of FIG. 1 where a first aperture is opened;

FIG. 8D depicts the biopsy device of FIG. 1 where a first sample is cut;

FIG. 8E depicts the biopsy device of FIG. 1 where a second aperture is opened; and

FIG. 8F depicts the biopsy device of FIG. 1 where a second sample is cut.

### DETAILED DESCRIPTION OF THE ILLUSTRATED EMBODIMENTS

In one embodiment, a biopsy device having a needle is provided. The needle disclosed herein comprises at least two apertures, each of which interacts with a cutter, and each cutter operates independently. By using the needle disclosed herein, a surgeon or other medical professional can obtain two or more biopsy specimen without the need to reinsert the needle. The number of device manipulation, and therefore, the time needed to complete the procedure, is thus cut down significantly. For purposes of illustration, the use of the embodiments of the needle disclosed herein is described with examples of various biopsy devices. However, those of ordinary skill in the art recognize that the embodiments of the needle disclosed herein can be used with any of the known biopsy devices, for example, those disclosed in U.S. Pat. Nos. 5,989,196, 5,368,045, 5,573,008, 5,823,971, 6,165,136, 6,273,861, and 7,001,341, each of which is incorporated by reference herein in its entirety, including the drawings.

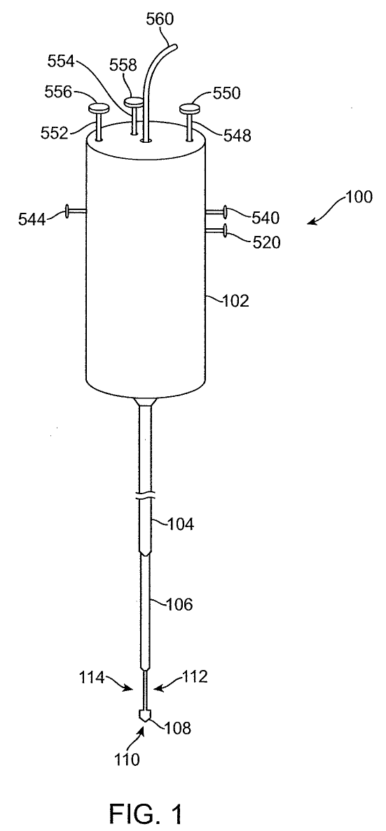

Referring to FIG. 1, an embodiment of a biopsy probe 100 includes a housing 102, a hollow cannula 104, and an elongated tube in the form of a needle 106. The needle 106 includes a sharp tip 108 at distal end 110. Proximal to the tip 108, the needle 106 includes at least two apertures 112 and 114, to be discussed in more detail below. The cannula 104 and needle 106 are axially movable with respect to each other. A vacuum tube 560, discussed in more detail below, is disposed coaxial to, and within, the cannula 104 and the needle 106.

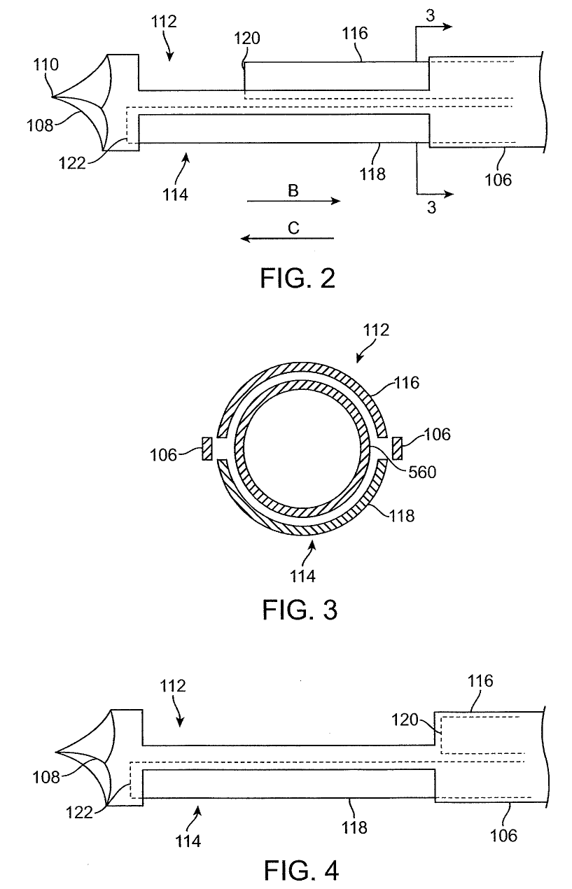

FIG. 2 shows the distal end of an embodiment of the needle 106, which terminates at a sharpened tip 108. The needle 106 has a diameter of between 14 gauge (approximately 1.6 mm in diameter) to 7 gauge (approximately 3.7 mm in diameter). The needle 106 is constructed from a biocompatible material that does not cause an allergic reaction when it comes into contact with human tissue. Preferably, the needle 106 is constructed from a metal, such as stainless steel or a titanium alloy, although in some embodiments, the needle 106 can be constructed from plastics. As seen in FIG. 3, the needle 106 is generally a hollow tube. In the illustrated embodiment, the tip 108 is sharp, such that it is capable of piercing tissue.

The needle 106 comprises at least two apertures at its distal end, substantially adjacent to the tip 108. FIG. 2 shows an embodiment of the needle 106 having two apertures 112 and 114. FIG. 3 is an end cross-sectional view of the needle 106 depicted in FIG. 2, taken along line 3-3 as viewed in the direction of the arrows. As shown in FIG. 3, the two apertures 112 and 114 radially oppose each other, and each of the apertures 112 and 114 circumferentially extends greater than 90° about the needle 106, and in particular, just a little less than 180° about the needle 106.

In some alternative embodiments, the needle 106 comprises three apertures, where each aperture extends over just a little less than 120° of the needle 106. Alternatively, the needle 106 comprises three apertures, where each aperture extends over just a little less than 90° of the needle 106. Each aperture 112 or 114 is of such length as to allow a sample large enough for biopsy to enter therethrough. For example, the length of each aperture can be 20 mm.

The needle 106 further comprises two cutters 116 and 118 slidably disposed in the needle 106. Addition cutters can be provided in the case where the needle 106 has more than two apertures. In the illustrated embodiment, each cutter 116 and 118 has a semicircular cross-section. Each cutter 116 and 118 is configured to move longitudinally along the length, or long axis, of the needle 106, and across a respective one of the apertures 112 and 114, in the direction of the arrow B in FIG. 2 from an extended position to a retracted position, and along the arrow C in FIG. 2 from a retracted position to an extended position. The distal ends 120 and 122 of cutters 116 and 118 are sharp and can cut tissue that is sucked into apertures 112 and 114 when cutters 116 and 118 are caused to move from their retracted position to their extended position, as discussed below.

In FIG. 2, the cutter 116 is shown in a partially retracted position, while in FIG. 4, the cutter 116 is shown in its fully retracted position. The cutter 118 is shown in its fully extended position in both FIGS. 2 and 4. In its fully retracted position, the cutter 116 is situated towards the proximal end of the needle 106, as shown in FIG. 4, such that the aperture 112 is fully open. In their fully extended position, the distal ends 120 and 122 of the cutters 116 and 118, respectively, are located at the distal end of the needle 106, near the tip 108, such that the apertures 112 and 114 are fully closed. Alternatively, when the cutters 116 and 118 are fully retracted, a portion of the distal ends 120 and 122, respectively, remains within the apertures 112 and 114, respectively. Likewise, when the cutters 116 and 118 are fully extended, a portion of the distal end 120 and 122 can remain within the apertures 112 and 114.

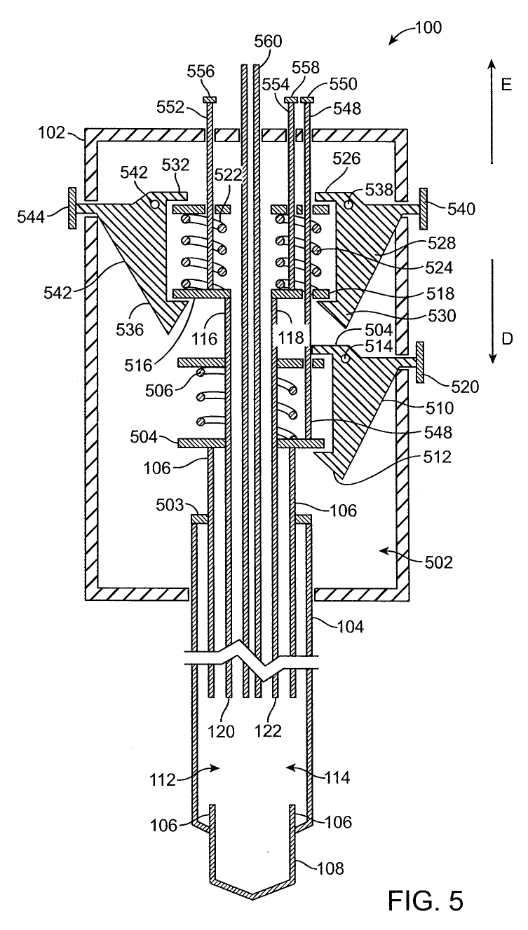

Referring to FIG. 5, the biopsy probe 100 comprises a vacuum coupling 560 located on the housing 102 for connecting to an external vacuum source (not shown). The needle 106 is located coaxially within the cannula 104. The tip 108 is protruding from cannula 104 to ease the penetration of the needle 106 into the target tissue. The cannula 104 extends shortly into the interior space 502 of the housing 102. The needle 106 extends through the cannula 104 and into the interior space 502 of the housing 102. The vacuum coupling 560 is in fluid communication with the lumen of the needle 106, such that when vacuum is applied to vacuum coupling 560, vacuum is created in the lumen of the needle 106. As a result, portions of the tissue in the proximity of the distal end of the needle 106 will prolapse into the apertures 112 and 114. Alternatively, the vacuum coupling 560 is in fluid communication with dual lumens within the needle 106, which respectively lead to the apertures 112 and 114.

The biopsy probe 100 includes a spring-loaded firing mechanism located within the housing 102. The collar 503, through which the needle 106 enters the cannula 104, comprises an air tight seal. The proximal end of the needle 106 attaches to the needle retaining collar 504, which is biased distally (arrow D) by spring 506. The spring 506 spirals around the tube defined by the cutters 116 and 118. The proximal end of the spring 506 rests against the rear lever 508 of the needle rocker arm 510 and is held in the compressed state by engagement of the needle retaining collar 504 by the latch portion 512 of the needle rocker arm 510. The needle rocker arm 510 is prevented from pivoting about the pin 514 (thereby releasing the needle retaining collar 504 and spring 506) when a selector switch 520 is in the distal position.

The proximal ends of cutters 116 and 118 extend through the needle 106, needle retaining collar 504, and spring 506 and are attached to the cutter retaining collars 516 and 518, respectively, which are biased distally (arrow D) by the springs 522 and 524, respectively. The proximal end of the spring 524 rests against the first cutter rear lever 526 of the first cutter rocker arm 528 and is held in the compressed state by engagement of the cutter retaining collar 518 by the latch portion 530 of the first cutter rocker arm 528. The first cutter rocker arm 528 is prevented from pivoting about the pin 538 (and thereby releasing cutter retaining collar 518 and spring 524) when the selector switch 540 is in the distal position.

Likewise, the proximal end of the spring 522 rests against the second cutter rear lever 532 of the second cutter rocker arm 534 and is held in the compressed state by engagement of the cutter retaining collar 516 by the latch portion 536 of the second cutter rocker arm 534. The second cutter rocker arm 534 is prevented from pivoting about the pin 542 (and thereby releasing the cutter retaining collar 516 and spring 522) when the selector switch 544 is in the distal position.

FIG. 5 shows both cutters 116 and 118 and needle 106 in their respective retracted positions. The needle 106 is moved to its retracted state by using rod 548 and handle 550. Handle 550 is pulled in the direction of arrow E, thereby causing the needle retaining collar 504 to also move in the direction of the arrow E until it is locked in place by the latch portion 512. Similarly, the cutters 116 and 118 are moved to their retracted position by using the rods 552 and 554 and handles 556 and 558, respectively. The handles 556 and 558 are pulled in the direction of arrow E, thereby causing the cutter retaining collars 516 and 518 to also move in the direction of the arrow E until they are locked in place by the latch portions 536 and 530, respectively.

The needle 106 is moved to its extended position by moving the selector switch 520 proximally, i.e., in the direction of arrow E, which causes the needle rocker arm 510 to pivot about the pin 514 and release the needle retaining collar 504. The spring 506 expands, forcing the needle 106 to move in the direction of arrow D. Alternatively, the needle 106 can be manually advanced by moving the entire probe 100 under ultrasound imaging guidance. The cutter 116 is moved to its extended position by moving the selector switch 544 proximally, i.e., in the direction of arrow E, which causes the cutter rocker arm 534 to pivot about the pin 542 and release the cutter retaining collar 516. The spring 522 expands, forcing the cutter 116 to move in the direction of arrow D. Similarly, the cutter 118 is moved to its extended position by moving the selector switch 540 proximally, i.e., in the direction of arrow E, which causes the cutter rocker arm 528 to pivot about the pin 538 and release the cutter retaining collar 518. The spring 524 expands, forcing the cutter 118 to move in the direction of arrow D. The spring force is sufficient to cause the cutters 116 and 118 to sever the tissue prolapsed within the apertures 112 and 114, as explained below.

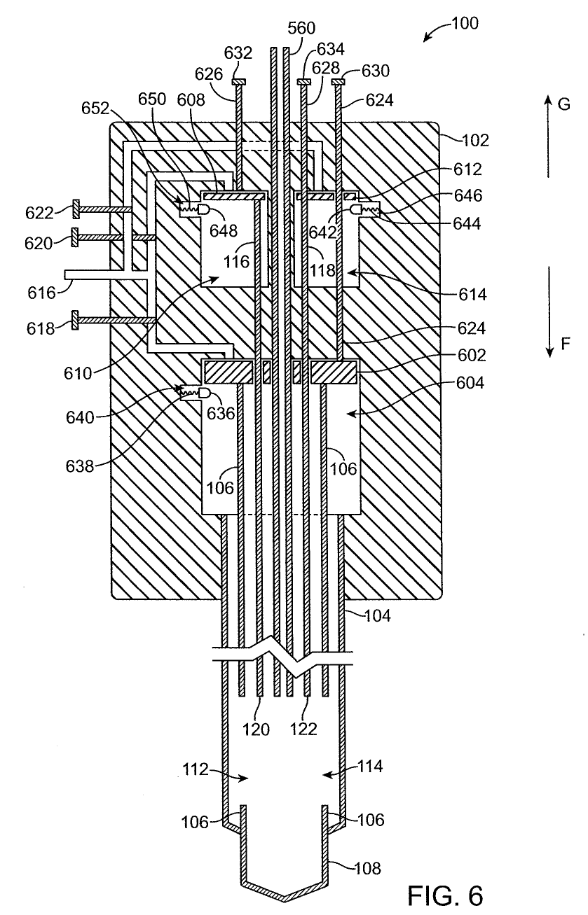

FIG. 6 shows a pneumatic firing mechanism of probe 100 located within the housing 102. At the proximal end of the needle 106 there is a needle piston collar 602, the outer walls and proximal surface of which cooperatively engage the inner walls of the needle piston housing portion 604. At the proximal end of the cutter 116 there is a cutter piston collar 608, the outer walls and proximal surface of which cooperatively engage the inner walls of the first cutter piston housing 610. Similarly, at the proximal end of the cutter 118 there is a cutter piston collar 612, the outer walls and proximal surface of which cooperatively engage the inner walls of the second cutter piston housing 614.

A fluid access coupling 616 provides mechanical connection to the interior of the housing 102 for the compressed fluid which provides the impelling force for the operation of the probe 100. Such a fluid may be any fluid capable of compression such that, upon the release of compressive forces, it expands into the inner structure of the housing 102, for example in the piston housings 604, 610, or 614, with sufficient force to drive the respective pistons 602, 608, or 612, in the distal direction. A preferred fluid would be compressed carbon dioxide gas (CO₂).

The valve 618 controls the flow of fluid from the fluid access coupling 616 to the needle piston housing portion 604. When the valve 618 is depressed, fluid flows into the needle piston housing portion 604 and causes the piston to move distally, i.e., in the direction of arrow F, thereby pushing the needle 106 in the distal direction. The force of fluid on the piston 602 is sufficient to cause the needle 106 to enter human tissue.

A notch 636 holds needle piston collar 602 in its fully retracted position. The notch 636 is held outside of the opening 640 by a spring 638. When the piston collar 602 is pushed distally, i.e., in the direction of arrow F, the notch 636 is forced to retract into the opening 640. When the piston collar 602 clears the notch 636, the spring 638 causes the notch 636 to return to its extended position. Similarly, when the piston collar 602 is returned to its retracted position by pulling on the handle 630, the piston collar 602 forces notch 636 to retract into the opening 640. When the piston collar 602 clears the notch 636, the spring 638 causes the notch 636 to return to its extended position, which then holds the piston collar 602 in its fully retracted position.

The valve 620 controls the flow of fluid from the fluid access coupling 616 to the first cutter piston housing portion 614. When the valve 620 is depressed, fluid flows into the first cutter piston housing portion 614 and causes the piston to move distally i.e., in the direction of arrow F, thereby pushing the cutter 118 at high speed in the distal direction to its fully expanded position. Similarly depressing valve 622 causes the cutter 116 to be pushed distally at high speed to its fully expanded position. The force of fluid on the pistons 608 and 612 is sufficient to cause the cutters 116 and 118 to sever tissue prolapsed within the apertures 112 and 114, as explained below.

The pistons 602, 608, and 612 can be moved proximally, i.e., in the direction of arrow G, by using the rods 624, 626, and 628, respectively, and the handles 630, 632, and 634, respectively. The handles 630, 632, and 634 can be pulled individually in the direction of arrow G, thereby causing the needle 106, cutter 116, and cutter 118, respectively, to move proximally and towards their respective retracted positions. The notches 642 and 648 and springs 644 and 650, located in the openings 646 and 652, respectively, work similarly to the notch 636 and spring 638 in the opening 640 to hold the piston collars 612 and 608, respectively, in their fully retracted position.

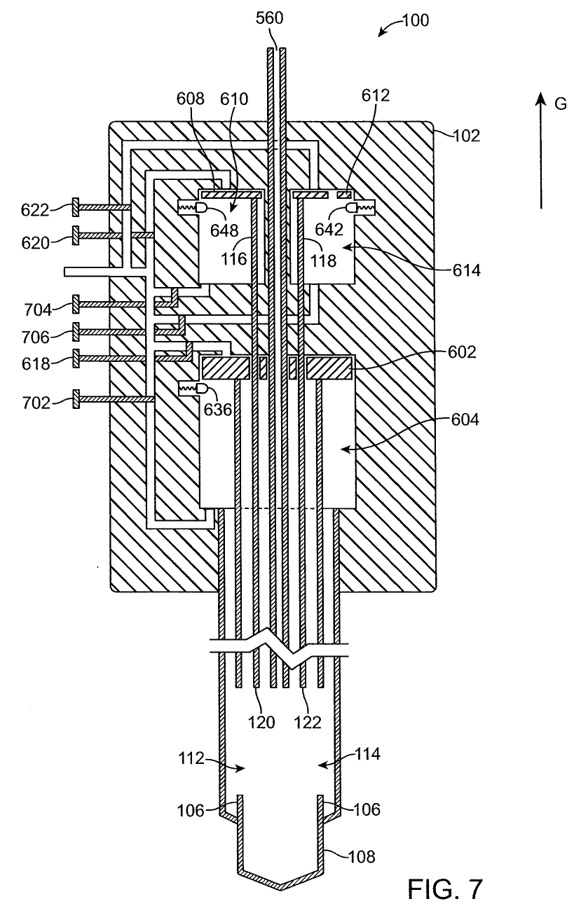

FIG. 7 shows another embodiment of the pneumatic firing mechanism, similar to that shown in FIG. 6. In the embodiment of FIG. 7, the pistons 602, 608, and 612 are moved in the proximal direction, i.e., in the direction of arrow G using compressed fluid. Starting with the piston collars 602, 608, and 612 held in their respective retracted positions by the notches 636, 648, and 642, respectively, the valve 618 is opened. Compressed fluid moves towards the housing portion 604, which causes the piston collar 602 to rapidly advance in a distal direction. When the piston collar 602 reaches the bottom of the housing portion 604, the valve 618 is closed. Similarly, the valves 620 and 622 are opened to cause the cutters 116 and 118 to rapidly advance in a distal direction. The valves 620 and 622 are then closed when the piston collars 602 and 612, respectively, reach the bottom of the housing portions 610 and 614, respectively. To retract the cutter 116, the valve 704 is opened, which causes compressed fluid to move the piston collar 608 in the proximal direction, i.e., in the direction of arrow G, until the piston collar 608 reaches the top of the housing portion 610 and is held in place by the notch 648. The piston collars 612 and 602, and thereby the cutter 118 and needle 106, respectively, are also retracted using a similar mechanism by opening valves 706 and 702, respectively. Alternatively, the embodiments shown in FIGS. 6 and 7 can function hydraulically.

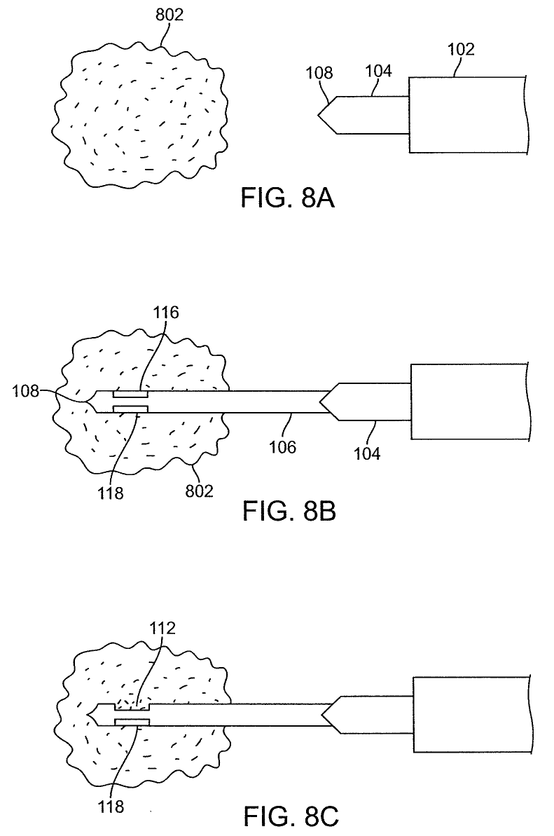

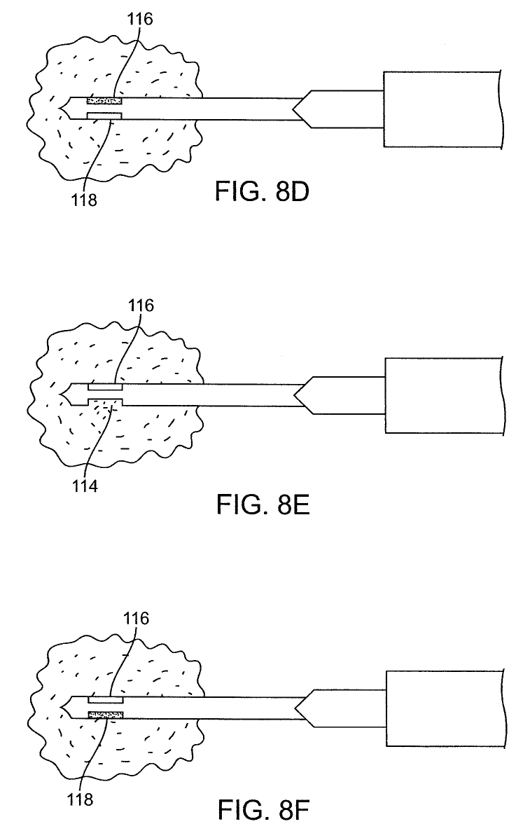

FIGS. 8A-8F illustrate a method of obtaining a biopsy sample from a tissue of a patient using the biopsy probe 100. As shown in FIG. 8A, the biopsy probe 100 and cannula 104 are advanced into the tissue with the needle 106 in its fully retracted position. The needle 106 is then fired in a distal direction, i.e., in the direction of arrows D and F, in FIGS. 5 and 6, respectively, and advanced into the tissue of interest 802, such as a tumor. Alternatively, the cannula 104 can be placed outside of the human body and the needle 106 can be advanced (see above) into the human body, if the tissue of interest 802 is of such depth as the length of the needle 106 alone is sufficient to advance the apertures 112 and 114 into the tissue 802. Alternatively, cannula 104 can be removed and needle 106 can be advanced manually under image guidance.

When the needle 106 is advanced into the tissue of interest 802, as shown in FIG. 8B, the cutters 116 and 118 are in their respective extended position, such that the apertures 112 and 114 are closed to the tissue 802. When the tissue of interest 802 is adjacent to the apertures 112 and 114, the cutter 116 is retracted (see above) to its fully retracted position, as shown in FIG. 8C. Vacuum is applied causing the tissue to prolapse into the aperture 112. Once a portion of the tissue prolapses within the aperture 112, the retracted cutter 116 is caused to rapidly advance (see above) towards the distal end of the needle 106, i.e., towards the extended position. The tissue portion that is in the aperture 112 is severed from the rest of the tissue. A biopsy sample is then obtained, which is then located inside of the needle 106, as shown in FIG. 8D. Vacuum is then applied and the biopsy sample is drawn out through the proximal end of the probe 100. Next, the cutter 118 is retracted to its fully retracted position (FIG. 8E). The same procedure as above is followed to sever a tissue sample that is located within the aperture 114 (FIG. 8F).

In some embodiments, after two biopsy samples are obtained through apertures 112 and 1 14, the needle 106 is rotated 90° at the same axial depth and the procedure is repeated to obtain two additional samples. It is understood that other rotational angles than 90° can be used, depending on the physician preference. Thus, an advantage of the present device and methods is that more than one sample, e.g., two, four, six, etc., can be obtained by inserting the device only once, thereby reducing the number of device manipulations, and the time for obtaining the desired number of samples, required to complete a biopsy procedure. It is possible to obtain 360° of contiguous tissue samples by rotating the device in place. Because of multiple opposing chambers on the same needle this results in a more efficient procedure. Notably, compared to a needle having a single cutter with two ports, a needle with two cutters obtains a larger volume of tissue from each side when each aperture is opened individually, in contrast to a smaller volume of tissue from each side when the apertures are opened simultaneously. In addition, the medical professional desires to get the best, most representative, sample that they can. When two apertures are open at the same time, if there are differential densities (stiffnesses) on each side of the needle, one side of the needle will fill more preferentially than the other.

The devices described herein can be used for obtaining a sample from any type of tissue, such as malignant tumors, benign tumors, muscle, nerve, lymph nodes, bone marrow, etc. At times, a physician may need to compare the morphology of normal tissue with that of a potentially malignant one. The physician can insert a device according to the present disclosure in the demarcation line between the potentially malignant tissue and the normal tissue. The potentially malignant tissue sample can be obtained using one of the cutters 116 or 118, while normal tissue sample can be obtained using the other of the cutters 116 or 118, without the need to move needle 106.

It is understood by those of skilled in the art that the steps in the above method can be practiced in various different orders. The listing of the steps in the particular order described above does not, and should not, limit the disclosed method to the particular disclosed order of steps. The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

What is claimed:

1. A tissue sampling device, comprising:
an elongated tube having a distal end with at least two apertures; and
at least two cutters disposed within the elongated tube, wherein each cutter is configured to be independently displaced longitudinally within elongated tube and across a respective one of the apertures to sever tissue prolapsed within the respective aperture.

2. The tissue sampling device of claim 1, wherein the at least two apertures includes only two apertures, and the at least two cutters includes only two cutters.

3. The tissue sampling device of claim 2, wherein the apertures radially oppose each other.

4. The tissue sampling device of claim 2, wherein each aperture circumferentially extends about the elongated tube greater than ninety degrees.

5. The tissue sampling device of claim 2, wherein each cutter has a semi-circular cross-section.

6. The tissue sampling device of claim 1, further comprising a firing mechanism configured to rapidly advance each cutter towards the distal end of the elongated tube and across the respective aperture to sever the prolapsed tissue.

7. The tissue sampling device of claim 6, wherein the firing mechanism is a pneumatic mechanism.

8. The tissue sampling device of claim 6, wherein the firing mechanism is a spring-loaded mechanism.

9. The tissue sampling device of claim 1, further comprising a cannula in which the elongated tube is coaxially housed.

10. The tissue sampling device of claim 1, wherein the elongated tube has one or more lumens in fluid communication with the apertures, and further comprising a vacuum port in fluid communication with the one or more lumens.

11. The tissue sampling device of claim 1, wherein the elongated tube has a size within the range of 14 gauge and 7 gauge.

12. The tissue sampling device of claim 1, wherein the elongated tube is a needle.

13. A method of obtaining a tissue sample using a biopsy probe, the biopsy probe comprising a first aperture, a second aperture, a first cutter, and a second cutter, the method, comprising:
inserting the biopsy probe in tissue;
receiving a first prolapsed portion of the tissue into the first aperture;
longitudinally displacing the first cutter across the first aperture to sever the first prolapsed tissue portion within the first aperture;
receiving a second prolapsed portion of the tissue into the second aperture; and
longitudinally displacing the second cutter independently from the first cutter across the second aperture to sever the second prolapsed tissue portion within the second aperture.

14. The method of claim 13, wherein the biopsy probe is inserted in the tissue while the first and second cutters respectively close the first and second apertures.

15. The method of claim 14, wherein the first and second cutters are independently longitudinally retracted to open the first and second apertures, thereby allowing the first and second prolapsed tissue portions to be received into the first and second apertures.

16. The method of claim 13, wherein the first and second prolapsed tissue portions are respectively vacuumed into the first and second apertures.

17. The method of claim 13, wherein the first and second cutters are rapidly displaced across the first and second apertures to sever the respective first and second prolapsed tissue portion.

18. The method of claim 13, wherein the first and second severed tissue portions are aspirated through the biopsy probe.

19. The method of claim 13, further comprising:
rotating the biopsy probe within the tissue;
receiving a third prolapsed portion of the tissue into the first aperture;
longitudinally displacing the first cutter across the first aperture to sever the third prolapsed tissue portion within the first aperture;
receiving a fourth prolapsed portion of the tissue into the second aperture; and
longitudinally displacing the second cutter independently from the first cutter across the second aperture to sever the fourth prolapsed tissue portion within the second aperture.

20. The method of claim 19, wherein the biopsy probe is rotated 90°.

21. The method of claim 13, wherein the tissue is malignant tissue.
